(19) United States
(12) Patent Application Publication
(10) Pub. No.: US 2003/0054043 A1
(43) Pub. Date: Mar. 20, 2003

(54) Thixotropic oil based vehicle for pharmaceutical compositions

(76) Inventor: Martin Kuentz, Muttenz (CH)

Correspondence Address: HOFFMANN-LA ROCHE INC., PATENT LAW DEPARTMENT, 340 KINGSLAND STREET, NUTLEY, NJ, 07110

(21) Appl. No.: 10/234,722

(22) Filed: Sep. 4, 2002

(30) Foreign Application Priority Data
Sep. 10, 2001 (EP) 01121545.6

(51) Int. Cl. A61K 9/14

(52) U.S. Cl. 424/489

(57) ABSTRACT

The present invention relates to a novel thixotropic oily vehicle comprising between about 0.2% to about 5% (w/w) of a colloidal silica and between about 0.2% to about 5% (w/w) of a hydrophilic polymer in an edible oil. The interaction between the hydrophylic polymer and the colloidal silica in the above concentration ranges confers thixotropy and a low viscosity under shear on the solution. The invention also relates to capsules filled with the above thixotropic solution used as a fill mass.

### FIELD OF INVENTION

The present invention is directed to pharmaceutical compositions and more particularly to a thixotropic oily vehicle with reduced levels of low density excipient useful as a fill mass for thermally labile pharmaceutically active compounds with low aqueous solubility.

### BACKGROUND

The filling of liquid and semi-solid fill masses into capsules is widespread in the pharmaceutical industry. The use of hard gelatin capsules has become increasingly important because of characteristics that make this dosage form even more preferred than that based on the soft gelatin technology. For example, hard gelatin shells are less sensitive towards heat and humidity and their permeability to oxygen is considerably lower than that of soft gelatin shells. Accordingly, hard gelatin capsules can be stored more easily and for a longer period of time without risking to damage the active compounds which they contain (see e.g. “Liquid Filled and Sealed Hard Gelatin Capsules”, E. T. Cole, Bulletin Technique Gattefossé, 1999, p.70).

The use of hard gelatin capsules in the pharmaceutical industry is reviewed for instance in “Liquid Filling of Hard Gelatin Capsules: A New Technology for Alternative Formulations”, W. J. Bowtle, Pharm. Technology Europe October 1998, pp. 84-90.

The feasibility of using capsules as unit dose for administering nutrients or pharmaceutical active ingredients depends on the flow behavior of the fill mass which has to be encapsulated. Ideally, the fill mass should be liquid during the filling process while it should solidify or become a gel once encapsulated.

It is advantageous that solidification or gelling of the fill mass occurs since, in this way, a final sealing step of the capsule shell can be avoided. For suspensions, a gelification with a relatively high yield point (i.e. the critical stress to induce plastic deformation of the material, measured in Pa) is important to prevent re-liquefaction of the fill mass by accidental shaking of the capsules during e.g. transportation. Accidental re-liquefaction of the fill mass after encapsulation can cause settling and caking of suspended active drug particles, thus potentially decreasing dissolution and possibly also the bioavailability of the active drug.

### SUMMARY

The present invention relates to a novel thixotropic oily vehicle comprising a relatively low amount of colloidal silica and to a fill mass containing this vehicle. Furthermore, the present invention is directed to capsules, in particular hard gelatin capsules, filled with the above fill mass.

The oily vehicle of the present invention contains a reduced amount of colloidal silica relative to the effect seen, has a relatively elevated yield point, a high degree of thixotropy and a low viscosity under shear. The reduced amount of colloidal silica is significant, reducing the bulk volume of the capsule filling mixture when it is processed on a production scale below that that would otherwise be expected.

There is an unexpected interaction between the hydrophylic polymer and the colloidal silica in the concentration ranges of the invention that results in an adequately thixotropic capsule fill mixture that has a low viscosity under shear and a relatively low colloidal silica content.

### DETAILED DESCRIPTION

The term “capsule” encompasses hard and soft shell capsules which are preferably used to orally administer nutrients or pharmaceutically active ingredients to individuals. Such capsules are soluble under physiological conditions, digestible or permeable. The capsule shells are usually made of gelatin, starch, or other suitable physiologically acceptable macromolecular materials in form of gels. Examples thereof are soft gelatin capsules, hard gelatin capsules and Hydroxy Propyl Methyl Cellulose (HPMC) capsules.

The term “fill mass” defines one or more active compounds and/or nutrients and (possibly) suitable additives dissolved in a pharmaceutically acceptable vehicle. An ideal fill mass is one that is readily delivered into a capsule and, once delivered becomes substantially solid, thus substantially preventing separation of the active ingredients and providing a unit dose with adequate shelf storage stability.

The term “vehicle” means an inert medium in which a medicinally active agent is administered.

A fill mass with ideal flow performance can be obtained by application of sufficient heat to melt a waxy formulation during filling or by providing a so-called thixotropic system. Thixotropy is a property of certain solids or gels, which liquefy when subjected to shear forces and then solidify again when left standing. A thixotropic transformation, i.e. solid/liquid/solid, does not involve application of heat and thus is especially suitable for thermolabile active pharmaceutical substances. The absence of a heating phase for a thixotropic transformation is also favorable for suspensions having sparingly soluble active drug components whereby increased drug solubility as a result of heating may result in a precipitation of the sparingly soluble drug upon cooling, thus potentially effecting the bioavailability and shelf storage stability.

The particular characteristics of thixotropic systems in the context of pharmaceutical fill masses are e.g. highlighted in “The filling of molten and thixotropic formulations into hard gelatin capsules”, S. E. Walker, J. A. Ganley, K. Bedford and T. Eaves, J. Pharm.Pharmacol. 32, 1980, pp. 389-393.

On the other hand, many substances obtained from modern drug discovery have bioavailability problems often exhibiting a sufficiently low aqueous solubility thereby necessitating formulation in oily (apolar) vehicles. Unfortunately, there are only few excipients that induce thixotropic behavior in oil based systems. The most significant of these excipients is silicon dioxide in the form of colloidal silica. These colloidal silica formulations provide thixotropy in oil based systems with a convenient yield point (>2-4 Pa) at concentrations between about 4 to about 10% (w/w) depending on the polarity of the oil.

The viscosity under shear of the thixotropic vehicle, which is measured at a defined shear rate, must be enough low (<300 mPa s) to enable filling of highly concentrated suspensions into capsules, where the viscosity is often the limiting factor of the technical feasibility. However, suspensions with a high amount of solid phase have to be processed to substantially eliminate the possibility of widevariance of the drug load range in each unit of the final dosage form.

It is furthermore desirable to keep the concentration of colloidal silica in the fill mass as low as possible since this colloidal powder has exceptionally low density (≈0.03 g/cm³) and is potentially harmful upon inhalation. The use of this colloidal silica on an industrial scale thus may raise several practical problems and may endanger the health of the technicians who work with it.

The problem at the root of the present invention is therefore to provide a thixotropic oily vehicle containing as little colloidal silica as possible that has both a high yield point (>4 Pa) and a low viscosity under shear (<300 mPa s).

This problem is solved, according to the present invention, by providing a thixotropic oily vehicle comprising between about 0.2% to about 5% (w/w) of a colloidal silica and between about 0.2% to about 5% (w/w) of a hydrophilic polymer. In the formulation of the invention, an unexpected interaction is seen between the several components in the preferred concentration ranges.

The positive effects of this interaction are quite surprising and unexpected. In fact, although it is known that additives may improve the thickening performance of the colloidal silica dioxide (see e.g. Degussa's Technical Bulletin No. 23: “Aerosil® as a Thickening Agent for Liquid Systems”, 1989, pp. 22-24) it is to be expected that the addition of a hydrophilic polymer leads to a phase separation in the apolar oily environment, rather than a homogenous colloidal system. However, in the concentration ranges of the present invention, the interaction of the colloidal silica surface with the hydrophilic polymer builds a coherent structure that unexpectedly provides the desired flow performance for liquid-fill systems.

When left standing, the composition of the present invention preferably has the visual appearance of a transparent oily gel.

According to a preferred embodiment of this invention, the colloidal silica is chosen from the group consisting of a fumed hydrophilic colloidal silica with a surface area of 200 square meters per gram (M²/g), a fumed hydrophilic colloidal silica with a surface area of 300 M²/g, and a fumed colloidal silica with a surface area of 300 M²/g rendered hydrohobic by treatment with hexamethyl disilizane. Suitable fumed colloidal silica having these preferred properties are, respectively, Aerosil® 200, Aerosil® 300 and Aerosil® R812 (all available from Degussa AG, Frankfurt) with the most preferred colloidal silica being a hydrophilic fumed colloidal silica with a surface area of 200 M²/g, e.g., Aerosil® 200 or the like. In the oily thixotropic vehicle of the invention, the colloidal silica is preferably used in a concentration between about 0.5% to about 3% (w/w) and, still more preferably, in a concentration between about 1% to about 2% (w/w).

A hydrophilic polymer used in the thixotropic oily vehicle according to the present invention is chosen from the group consisting of polyethers and polyalcohols. Suitable polyethers and polyalcohols include, but are not limited to, polyethylene glycols, polypropylene-polyethylene glycols and polyvinylalcohols. Polyethylene glycols having a molecular weight equal to or less than about 400 g/mol are preferred. Examples thereof are polyethylene glycol with a molecular weight about 200 g/mol, polyethylene glycol with a molecular weight about 300 g/mol and polyethylene glycol with a molecular weight about 400 g/mol. Most preferred is the polyethylene glycol with a molecular weight about 300 g/mol.

The hydrophilic polymer is preferably present in the thixotropic oily vehicle of the invention in a concentration between about 0.5% to about 4% (w/w) and, more preferably, in a concentration between about 1% to about 3% (w/w).

As stated above, the thixotropic oily vehicle of the present invention is suitable for the preparation of liquid-filled capsules which are intended for oral drug delivery. It is particularly suitable for active compounds whose oral bioavailability and/or chemical stability can be improved by a lipidic or oil based formulation rather than by a conventional dosage form with an aqueous based formulation. The special pharmacokinetic profile of certain active compounds can be a further reason to use a lipidic vehicle as dispersing medium. Examples of such active compounds where oil based formulations are useful include esters, lactones, retinoids, steroids, dihydropyridins and 4-phenylpyridin derivatives. Particularly, the thixotropic oily vehicle of the present invention is preferred for active compounds selected from the group of the 4-phenylpyridine derivatives consisting of:

2-(3,5-bis-trifluoromethyl-phenyl)-N-methyl-N-(6-morpholin-4-yl-4-o-tolyl-pyridin-3-yl)-isobutyramide;

2-(3,5-bis-trifluoromethyl-phenyl)-N-methyl-N-[6-(4-methyl-piperazin-1-yl)-4-o-tolyl-pyridin-3-yl]-isobutyramide; and

2-(3,5-bis-trifluoromethyl-phenyl)-N-[4-(2-chloro-phenyl)-pyridin-3-yl]-N-methyl-isobutyramide.

The above three compounds, whose synthesis may be found in EP-A-1035115, are characterized by valuable therapeutic properties. They are highly selective antagonists of the Neurokinin 1 (NK-1, substance P) receptor. Substance P is a naturally occurring undecapeptide belonging to the tachykinin family of peptides, the latter being so-named because of their prompt contractile action on extravascular smooth muscle tissue.

The oily component of the vehicle according to the present invention consists of an edible oil which can be chosen from the natural and semi-synthetic vegetable mono-, di- or triglycerides. Preferred are pharmaceutical grade triglyceride oils such as corn oil, peanut oil, olive oil, castor oil, or a middle chain triglyceride oil such as caprylic/caproic glyceride (Miglyol, as available from Degussa-Huls is well-suited) or mixtures thereof. Most preferred is the middle chain triglyceride oil (Miglyol).

The present invention is also directed to a process for preparing a thixotropic oily vehicle as described above, which process comprises mixing, in an edible oil as defined above, between about 0.2% to about 5% (w/w) of a colloidal silica with between about 0.2% to about 5% (w/w) of a hydrophilic polymer.

A further embodiment of the present invention consists of a fill mass comprising a thixotropic oily vehicle as described above and a therapeutically effective amount of one or more pharmaceutically active ingredients.

A still further embodiment of the present invention is directed to pharmaceutical unit dose wherein a fill mass as described above is encapsulated in an edible capsule. In a preferred embodiment, the capsule is made of gelatin and, still more preferably, of hard gelatin.

The present invention is further described by the following non-limiting examples. Table 1 shows the viscosity under a defined shear and the yield point of the exemplified oily vehicles, as well as of comparative oily vehicles which do not include a hydrophilic polymer.

The rheological characterization was performed using a controlled stress instrument Carri-Med CSL 500 equipped with a cone and plate system (6 cm diameter and 2° angle). The viscosity was determined at a shear rate of 100 s⁻¹ and a temperature of 25° C. on the “down-curve” of the hysteresis flow curve. On the other hand, the “up-curve” was used to extrapolate the yield point according to the Casson model (“Das Rheologie Handbuch für Anwender von Rotations- und Oszillations-Rheometern”, T. Mezger, Vincentz, 2000, p.54).

### PREPARATIONS OF THE COMPOSITION

### Example 1

2.0 g Aerosil® 200 were exactly weighted and dispersed with a mixer (Type Bamix® (Switzerland), level 2 during 30 seconds) in 96.0 g of Miglyol 812 (middle chain triglyceride). 2.0 g of fluid polyethylene glycol with a molecular weight about 400 g/mol were added to and mixed with the above suspension (Bamix, level 2 during 45 seconds). The so obtained thixotropic vehicle was finally put under vacuum to remove the incorporated air.

### Example 2

The procedure of Example 1 was repeated with the following composition:

| | | |
|---|---|---|
| 1.5 g | Aerosil ® | 200 |
| 2.0 g | Polyethylene glycol | 300 |
| 96.5 g | Miglyol | 812 (middle chain triglyceride) |

### Example 3

The procedure of Example 1 was repeated with the following composition:

| | | |
|---|---|---|
| 2.0 g | Aerosil ® | 200 |
| 2.5 g | Polyethylene glycol | 300 |
| 95.5 g | Miglyol | 812 (middle chain triglyceride) |

### Example 4

The procedure of Example 1 was repeated with the following composition:

| | | |
|---|---|---|
| 1.5 g | Aerosil ® | 200 |
| 2.0 g | Polyethylene glycol | 300 |
| 96.5 g | Peanut oil | |

### Example 5

The procedure of Example 1 was repeated with the following composition:

| | | |
|---|---|---|
| 5.0 g | 2-(3,5-bis-trifluoromethyl- | |
| | phenyl)-N-methyl-N-(6- | |
| | morpholin-4-yl-4-o-tolyl- | |
| | pyridin-3-yl)-isobutyramide. | |
| 1.5 g | Aerosil ® | 200 |
| 1.0 g | Polyethylene glycol | 300 |
| 92.5 g | Miglyol | 812 (middle chain triglyceride) |

### Example 6

The procedure of Example 1 was repeated with the following composition:

| | | |
|---|---|---|
| 5.0 g | 2-(3,5-bis-trifluoromethyl- | |
| | phenyl)-N-methyl-N-(6- | |
| | morpholin-4-yl-4-o-tolyl- | |
| | pyridin-3-yl)-isobutyramide. | |
| 1.5 g | Aerosil ® | 200 |
| 2.0 g | Polyethylene glycol | 300 |
| 91.5 g | Miglyol | 812 (middle chain triglyceride) |

### Example 7

The procedure of Example 1 was repeated with the following composition:

| | | |
|---|---|---|
| 5.0 g | 2-(3,5-bis-trifluoromethyl- | |
| | phenyl)-N-methyl-N-(6- | |
| | morpholin-4-yl-4-o-tolyl- | |
| | pyridin-3-yl)-isobutyramide. | |
| 1.5 g | Aerosil ® | 200 |
| 3.0 g | Polyethylene glycol | 300 |
| 90.5 g | Miglyol | 812 (middle chain triglyceride) |

### Example C1 (Comparative)

The procedure of Example 1 was repeated with the following composition:

| | | |
|---|---|---|
| 2.0 g | Aerosil ® | 200 |
| 98.0 g | Miglyol | 812 (middle chain triglyceride) |

### Example C2 (Comparative)

The procedure of Example 1 was repeated with the following composition:

| | | |
|---|---|---|
| 5.0 g | Aerosil ® | 200 |
| 95.0 g | Miglyol | 812 (middle chain triglyceride) |

### Example C3 (Comparative)

The procedure of

### Example 1 was repeated with the following composition:

| | | |
|---|---|---|
| 6.0 g | Aerosil ® | 200 |
| 94.0 g | Miglyol | 812 (middle chain triglyceride) |

### Example C4 (Comparative)

5.0 g 2-(3,5-bis-trifluoromethyl-phenyl)-N-methyl-N-(6-morpholin-4-yl-4-o-tolyl-pyridin-3-yl)-isobutyramide.

1.5 g Aerosil® 200

93.5 g Miglyol 812 (middle chain triglyceride)

TABLE 1
Rheological Characterization

| | Amount of | Amount of | Viscosity | |
|---|---|---|---|---|
| | Aerosil ® 200 | polyethylene | (100 s⁻¹/25° C.) | Yield point |
| Ex. | (% w/w) | glycol (% w/w) | (mPa s) | (Pa) |
| 1 | 2.0 | 2.0 | 55 | 8.30 |
| 2 | 1.5 | 2.0 | 137 | 7.13 |
| 3 | 2.0 | 2.5 | 207 | 17.08 |
| 4 | 1.5 | 2.0 | 249 | 7.23 |
| 5 | 1.5 | 1.0 | 205 | 5.01 |
| 6 | 1.5 | 2.0 | 149 | 4.67 |
| 7 | 1.5 | 3.0 | 135 | 4.68 |
| C1 | 2.0 | — | 56 | 0.14 |
| C2 | 5.0 | — | 201 | 4.00 |
| C3 | 6.0 | — | 349 | 9.07 |
| C4 | 1.5 | — | 59 | 0.11 |

As it can be seen from Table 1, the addition of a hydrophilic polymer (polyethylene glycol) enables a decrease in the amount of colloidal silica necessary to confer to the oily vehicle a sufficiently high yield point (at least 4 Pa), by keeping the viscosity under shear below 300 mPa s. Without the addition of the hydrophilic polymer, yield points above 4 can be obtained only at Aerosil® concentrations of 5% (w/w) or more.

If Example 2 and Example C2 are compared, it can be seen that the addition of 2% (w/w) of polyethylene glycol enables a decrease in the amount of Aerosil® by a factor 3.33 (w/w) and still provides an almost doubled yield point (7.13 vs. 4 Pa) and a lower viscosity under shear (137 vs. 201 mPa s).

Other comparisons from Table 1 between the vehicles according to the present invention and the conventional ones (e.g. Ex 1 with Ex C1) demonstrate that, at a Aerosil® concentration of 2%, the addition of a hydrophilic polymer enables a strong increase in the yield point (0.14 vs. 8.30 Pa).

1. A vehicle for a pharmaceutical composition comprising between about 0.2% to about 5% (w/w) of a colloidal silica and between about 0.2% to about 5% (w/w) of a hydrophilic polymer in an edible oil.

2. The vehicle according to claim 1, wherein the colloidal silica is present in a concentration between about 0.5% to about 3% (w/w).

3. The vehicle according to claim 2, wherein the colloidal silica is present in a concentration between about 1% to about 2% (w/w).

4. The vehicle according to claim 1, wherein the colloidal silica is selected from the group consisting of a hydrophilic colloidal silica with a surface area of 200 M²/g, a hydrophilic colloidal silica with a surface area of 300 M²/g and a hydrophilic colloidal silica with a surface area of 300 M²/g rendered hydrophobic by treatment with hexamethyldisilizane.

5. The vehicle according to claim 4, wherein the colloidal silica is a hydrophilic colloidal silica with a surface area of 200 M²/g.

6. The vehicle according to claim 1, wherein the hydrophilic polymer is present in a concentration between about 0.5% to about 4% (w/w).

7. The vehicle according to claim 6, wherein the hydrophilic polymer is present in a concentration between about 1% to about 3% (w/w).

8. The vehicle according to claim 1, wherein the hydrophilic polymer is selected from the group consisting of polyethers and polyalcohols.

9. The vehicle according to claim 8, wherein the hydrophilic polymer is a polyethylene glycol having a molecular weight less than about 400 g/mol.

10. The vehicle according to claim 9, wherein the hydrophilic polymer is a polyethylene glycol with a molecular weight of about 300 g/mol.

11. The vehicle according to claim 1, wherein the edible oil is chosen from the group consisting of natural and semi-synthetic vegetable mono-, di- and triglycerides.

12. The vehicle of claim 11, wherein the edible oil is a triglyceride oil.

13. The vehicle of claim 12, wherein the triglyceride oil is selected from the group consisting of corn oil, peanut oil, olive oil, castor oil, and middle chain triglyceride oil.

14. The vehicle of claim 13, wherein the triglyceride oil is caprylic/caproic triglyceride oil.

15. A process for preparing a vehicle according to claim 1 comprising mixing, in an edible oil, between about 0.2% to about 5% (w/w) of a colloidal silica with between about 0.2% to about 5% (w/w) of a hydrophilic polymer.

16. A fill mass comprising a vehicle according to claim 1 and a therapeutically effective amount of pharmaceutically active substance.

17. A pharmaceutical unit dose comprising a fill mass according to claim 16, encapsulated in an edible capsule.

18. The pharmaceutical unit dose of claim 17, wherein the capsule is made of gelatin.

19. The pharmaceutical unit dose of claim 18, wherein the capsule is made of hard gelatin.

20. A process for preparing a thixotropic oily vehicle for a pharmaceutical composition comprising mixing, in caprylic/caproic triglyceride oil, between about 0.2% to about 5% (w/w) of a colloidal silica with a surface area about 200 M²/g, with between about 0.2% to about 5% (w/w) of a polyethylene glycol with a molecular weight about 300 g/mol.
